(19) United States
(12) Patent Application Publication
(10) Pub. No.: US 2024/0373940 A1
(43) Pub. Date: Nov. 14, 2024

(54) INTERCHANGEABLE MOUTHPIECE FOR AN INHALER, CARTRIDGE SYSTEM, INHALER SYSTEM AND METHOD FOR DETERMINING A SYSTEM STATE OF AN INHALER

(71) Applicant: KÖRBER TECHNOLOGIES GMBH, Hamburg (DE)

(72) Inventor: Michael Joseph CAPONE, HAMBURG (DE)

(21) Appl. No.: 18/286,482

(22) PCT Filed: Apr. 12, 2022
(86) PCT No.: PCT/EP2022/059775

§ 371 Date: Oct. 11, 2023

(30) Foreign Application Priority Data
Apr. 14, 2021 (DE) 10 2021 109 301.5

(51) Int. Cl. A24F 40/51; A24F 7/02; A24F 40/10; A24F 40/42; A24F 40/48; A24F 40/53; A24F 40/65; G16H 20/13

(52) U.S. Cl. CPC A24F 40/51; A24F 7/02; A24F 40/10; A24F 40/42; A24F 40/48; A24F 40/53; A24F 40/65; G16H 20/13

(57) ABSTRACT

An interchangeable mouthpiece for an inhalator, in particular for an electronic cigarette product, a conventional cigarette product or a medical inhaler, comprises a housing which forms an interior space and has an air inlet opening and an air outlet opening, an adapter by means of which the mouthpiece is connectable to a mouth-side end of the inhalator so that the air inlet opening is flow-coupled to the inhalator in a connected state, and a data acquisition device which at least comprises a sensor, a data memory, a processor and an energy storage unit, with a flow channel being formed in the interior of the housing as a result of a separation and flow-connecting the air inlet opening to the air outlet opening, and with the data acquisition device being configured to store, in the data memory, a data record comprising at least an operational parameter depending on a fluidic condition within the flow channel.

The present invention relates to an interchangeable mouthpiece for an inhaler, in particular for an electronic cigarette product, a conventional cigarette product or a medical inhaler, comprising a housing which forms an interior and comprises an air inlet opening and an air outlet opening, an adapter, via which the mouthpiece is connectable to a mouth-side end of the inhaler such that the air inlet opening is fluidically coupled to the inhaler in a connected state, and a data acquisition device comprising at least a sensor, a data storage device, a processor, and an energy storage device. Furthermore, the invention relates to a corresponding cartridge system, an inhaler system, and a method for determining a system state of an inhaler.

Inhalers, be it medical inhalers, electronic cigarettes or conventional cigarettes, must meet the highest quality standards. To ensure this, the manufacturing processes of such products are constantly optimized. In this way, efforts are made to offer the end customer a product that can be operated at an ideal operating point.

Furthermore, it is desirable to be able to operate the inhaler under optimal conditions throughout its life cycle.

For this purpose, WO 2020/023547 A1 proposes an inhaler with a control unit adapted, for example, to precisely adjust the dose of a medium to be inhaled.

Furthermore, US 2016/0331025 A1 discloses a method in which usage data of an electronic vaporization device are sent to a central server and there an evaluation of the usage data takes place so that a usage profile can be created, which is then in turn sent to the electronic vaporization device for adjustment of the same.

Furthermore, US 2016/0371437 A1 discloses a data acquisition device that can be coupled to a conventional cigarette or an electric cigarette. By means of the data acquisition device, usage data of the cigarette can be captured and transmitted to an external storage device.

Although the solutions known from the prior art offer possibilities for capturing usage data, these do not offer satisfactory solutions with respect to a reproducible capture of the data, in particular with respect to the least possible interference with the user during data capture.

It is the task of the invention to provide an interchangeable mouthpiece with which operating parameters of an inhaler can be captured more reliably. It is further the task of the invention to provide a correspondingly improved cartridge system, an inhalation system and a method for determining a system status.

The invention solves the task by the features of the independent claims. Further preferred embodiments of the invention can be found in the dependent claims, the figures and the accompanying description.

According to the invention, it is proposed that a flow channel is formed inside the housing by a partition, which fluidically connects the air inlet opening to the air outlet opening, wherein the data acquisition device is adapted to store a data set comprising at least one operating parameter on the data memory in dependence on a fluidic condition inside the flow channel.

By storing the data in dependence on the fluidic condition within the flow channel, a high data quality of the stored data set can be achieved. The invention has recognized that statistically relevant data for determining a system condition is generated only under certain fluid mechanical conditions. The mouthpiece therefore allows to store only the required data in an efficient manner. Furthermore, this measure allows the data set to be stored fully automatically without the user having to consciously intervene.

Due to the partition, the flow channel is fluidically separated from the rest of the interior, preferably the flow channel is hermetically separated from the rest of the interior by the partition. The formation of a separate flow channel inside the housing allows reproducible flow conditions to be created, so that the data acquisition device can save data sets that are comparable with each other on the data memory.

Furthermore, the separate flow channel allows for optimal flow guidance inside the housing, resulting in a reduction of flow resistance. Due to the minimized flow resistance, a user is not affected or influenced by the mouthpiece during an inhalation process. This is especially important because it is an interchangeable mouthpiece that is operated with a separate inhaler. The user is usually accustomed to the draw resistance of the actual inhaler. Due to the low flow resistance, the draw resistance perceived by the user differs only slightly from the draw resistance of the actual inhaler, so that the data generated by means of the mouthpiece are obtained under unbiased conditions.

The flow channel can, for example, be formed entirely by the partition. However, it is also possible, for example, for the flow channel to be formed only partially by the partition and for the further boundary of the flow channel to be formed by a part of the housing.

Preferably, the data acquisition device is also arranged within the housing. By arranging both the data acquisition device and the flow channel within the housing, a compact and easy-to-handle mouthpiece is created.

Preferably, the adapter is configured to be connected to an inhaler mouthpiece. This feature means that the inhaler does not have to be modified and/or disassembled to bring the mouthpiece into use. It is sufficient to connect the inhaler mouthpiece to the adapter, which can be done, for example, by simply plugging it on. In this way, any inhaler type can be easily and inexpensively retrofitted.

Preferably, the data acquisition device is adapted to detect an inhalation puff, i.e. a so-called puff, on the basis of the fluidic conditions in the flow channel. The application has recognized that, in particular, operating parameters generated during the inhalation puff or describing the inhalation process itself are suitable for drawing a conclusion about the system state of the inhaler connected to the mouthpiece.

Further preferably, the storage of the data set is done in dependence of the detection of an inhalation puff. It has been shown that one or more of the fluidic conditions in the flow channel that occur during an inhalation puff is a suitable condition to generate and store the data set. Preferably, exactly one data set is generated per detected inhalation puff. This condition has proven to be particularly advantageous for the automatic collection of relevant statistical data. Based on data obtained in this way, well-founded conclusions can be drawn about the technical condition of a system, for example the condition of the inhaler connected to the mouthpiece. Alternatively or additionally, for example, the exceeding or falling below of a temperature or a flow velocity within the flow channel can also be used as a fluidic condition in the flow channel.

Preferably, the data collection device comprises one or more of the following sensors: a pressure sensor for detecting an air pressure in the flow channel; a temperature sensor for detecting the temperature within the flow channel; an ambient temperature sensor for detecting the temperature in the environment of the mouthpiece; a GPS receiver for detecting the current position of the mouthpiece; an acceleration sensor for detecting the orientation of the mouthpiece in space; and/or a sensor for detecting the flow resistance in the flow channel. It has been shown that these sensors can be used to capture operating parameters in a particularly advantageous manner.

Preferably, one or more of these sensors are provided in addition to the pressure sensor for detecting the flow channel pressure. The air pressure inside the flow channel detected by means of the pressure sensor is preferably used to detect an inhalation puff. By means of the pressure sensor, the beginning and the end of an inhalation puff can be easily detected.

It is further proposed that the data acquisition device is adapted to provide the data set with a time stamp. By means of the time stamp, the captured operating parameters can be uniquely assigned in terms of time, so that the operating parameters can also be evaluated over time. Preferably, the time stamp comprises a date corresponding to the inhalation puff, the puff, and/or a time corresponding to the inhalation puff, for example the time at which the start of a puff is detected.

For generating the time stamp, the data acquisition device preferably comprises a real time clock (RTC), for example in the form of a hardware clock or a software clock, the time and date information of which can be used to provide the data sets with a time stamp.

It is further proposed that the stored data set comprises at least one of the following operating parameters: a mouthpiece ID; an inhalation puff ID; an inhalation puff duration (so-called puff duration); a temperature inside the flow channel, a temperature in the environment of the mouthpiece; orientation data allowing inference of the orientation of the mouthpiece in space; coordinates allowing inference of the position of the mouthpiece; flow resistance data allowing inference of the flow resistance in the flow channel. These operating parameters have proven to be particularly advantageous for subsequent evaluation to determine a system status of the inhaler. The temperature within the flow channel can be used to deduce the temperature of the aerosol generated, for example, by a vaporizer of the inhaler; thus, for example, conclusions can be drawn about the system state of the vaporizer.

For the purpose of this application, an ID means a unique identification code, for example comprising a letter and/or number code. This allows the other operating parameters to be unambiguously assigned to a specific mouthpiece and/or a specific inhalation puff, also known as a puff.

It is further proposed that the mouthpiece comprises a data interface for transmitting the at least one stored data set. Due to the data interface, evaluation of the operating parameters does not have to be performed by the mouthpiece itself, but can be outsourced to an external evaluation device. The data interface may comprise, for example, a contact interface, for example a USB interface, further for example in USB-C format. Through such an interface, a simple and inexpensive data connection can be established between the mouthpiece and an external evaluation device.

It is further proposed that the data interface comprises a wireless interface comprising a Bluetooth module, a mobile radio module and/or a WiFi module.

For example, the mobile radio interface may comprise a so-called 5G module. Of course, the mobile radio interface may also operate according to another common standard.

The Bluetooth interface can be used, for example, to transfer the at least one data set to a user terminal, to a smartphone or a laptop, by means of an application, so that the at least one data set can then be transferred to the external evaluation device by means of an internet connection; preferably, the at least one data set created can thus be stored in a cloud, which enables the user to access it regardless of location.

Preferably, the connection data for establishing the connection via the wireless interface is pre-stored. This means that the connection information is already stored when the mouthpiece is delivered, for example on the data memory. For example, the access data for the mobile radio module may be pre-stored on a SIM card or eSim.

Provided that the data interface comprises a mobile radio module, the mouthpiece can be operated as a stand-alone internet terminal, for example. A data set can be transmitted via the mobile radio module, for example directly after each inhalation puff, to an external evaluation device, for example to a server. Alternatively, the transmission can also take place at regular time intervals, for example daily or weekly, and/or when a predefined number of data sets, for example 50 or 100 data sets, have been generated and stored.

Preferably, the adapter is formed by a part of the housing. Further preferably, the adapter is formed by a receptacle into which the inhaler can protrude and in such a way that slipping out of the inhaler is prevented by a force closure between the mouthpiece and the inhaler. The inhaler mouthpiece is thus held firmly by the adapter. The outer contour of the inhaler therefore comprises a minimal oversize compared to the inner contour of the adapter. To ensure that the mouthpiece is also secured in the connected state during mobile use, the mouthpiece can comprise an additional securing element by means of which slipping of the inhaler out of the adapter is additionally prevented by a form fit.

It is further proposed that the air inlet opening is arranged on a first end face of the mouthpiece associated with the adapter, and the air outlet opening is arranged on a second end face of the mouthpiece facing away from the adapter. The flow channel thus extends between two preferably opposing end faces, so that the air outlet opening comprises a similar arrangement and orientation with respect to the mouthpiece as the inhaler's own outlet opening comprises with respect to the inhaler's own mouthpiece.

It is further proposed that the flow channel connects the air inlet opening in a direct path to the air outlet opening. By this measure, the flow resistance within the flow channel can be further reduced. Firstly, this means that measurements in the flow channel of the mouthpiece can be used to make even better conclusions about the system status of the inhaler. Secondly, for the user, this means that the draw behavior during the inhalation puff is adapted in the best possible way to the draw behavior of the inhaler.

It has proven advantageous if the data acquisition device is supported by a carrier, which is supported on the inner wall of the housing, and by an outer surface of the partition forming the flow channel. Such a mounting of the data acquisition device is advantageous because sensors can thus protrude into the flow channel in a structurally simple manner. Furthermore, such a bearing arrangement allows a compact design and a weight-saving structure of the mouthpiece, which is of crucial importance for the handling and acceptance of the additional mouthpiece.

Preferably, the data acquisition device comprises at least one circuit board on which the electrical components of the data acquisition device are arranged. Preferably, this circuit board is then connected to the carrier.

The task mentioned at the beginning is further solved by a cartridge system comprising a cartridge having a cartridge mouthpiece, a reservoir with a substance to be vaporized and an adding device for adding the vaporized substance into an air stream, wherein the cartridge system comprises a mouthpiece according to any one of claims 1 to 12, wherein the adapter of the mouthpiece is detachably connected to the cartridge mouthpiece. For example, the cartridge is adapted to form an inhaler together with further components, in particular an energy storage device. Preferably, the reservoir is a tank in which a liquid to be vaporized is stored, which is then vaporized by the adding device comprising a vaporizer. In an alternative embodiment, the reservoir may also contain a tobacco substance which is heated by an adding device comprising a heater so that certain ingredients of the tobacco substance are volatilized.

Preferably, the shape of the outer contour of the mouthpiece mimics the shape of the cartridge mouthpiece, even if the mouthpiece mimics that shape on a larger scale.

The aforementioned task is also solved by an inhaler system comprising an inhaler having an inhaler mouthpiece, a reservoir with a substance to be vaporized and an adding device for adding the vaporized substance into an air stream and an energy storage device, wherein the inhaler system comprises a mouthpiece according to any one of claims 1 to 12, wherein the adapter of the mouthpiece is detachably connected to the inhaler mouthpiece. Accordingly, the inhaler may also comprise the cartridge described above.

Preferably, the shape of the outer contour of the mouthpiece mimics the shape of the cartridge mouthpiece, even if the mouthpiece mimics that shape on a larger scale.

As an alternative to attaching or coupling the mouthpiece to an inhaler or cartridge, the mouthpiece can also be attached to a conventional tobacco cigarette by means of its adapter. The adapter is then attached to the filter of the conventional cigarette.

The aforementioned task is also solved by a method for determining a system state of an inhaler comprising the following method steps:
a) mounting a mouthpiece according to any one of claims 1 to 12 on an inhaler's own inhaler mouthpiece;
b) determining and storing at least one operating parameter for an inhalation puff as a data set on the data memory;
c) assigning a time stamp with the data set on the data storage device;
d) transmitting a plurality of stored data sets by means of a data interface to an external evaluation device; and
e) evaluation of the data sets by means of the external evaluation device.

Preferably, in method step b), a data set is generated for each detected inhalation puff and saved on the data memory.

Preferably, in process step c), the data set is already provided with the corresponding time stamp when the operating parameters are stored.

Preferably, steps a) to b) are repeated until sufficient data sets have been generated. Only then do process steps d) and e) follow.

Preferably, a large number of data sets, for example several hundred data sets, are transmitted to the external evaluation device in process step d). The external evaluation device can then be used to perform calculation steps which allow conclusions to be drawn about the system status of the inhaler which is or was connected to the mouthpiece via the adapter.

By way of illustration only, a simple example is used to explain how, for example, a parameter can be determined that allows conclusions to be drawn about the total operating time of a vaporizer of the inhaler: if the values of the inhalation puff duration stored in the individual data sets are added together, a value is obtained that represents the duration of all inhalation puffs. If, for example, a vaporizer is only activated during an inhalation puff, the total operating time of the inhaler can thus be determined relatively accurately. Furthermore, depending on the result, for example if a threshold value is exceeded or not reached, a request for action can be generated, for example cleaning of the vaporizer or another maintenance measure. This request for action can be transmitted to the user via a separate communication channel, for example. For example, transmission via a data connection to a user's smartphone is conceivable.

In a further process step, the proposed request for action can be carried out. If, for example, a maintenance measure is carried out on the inhaler, its system status can be improved.

The invention is explained below with reference to preferred embodiments with reference to the accompanying figures. Thereby shows

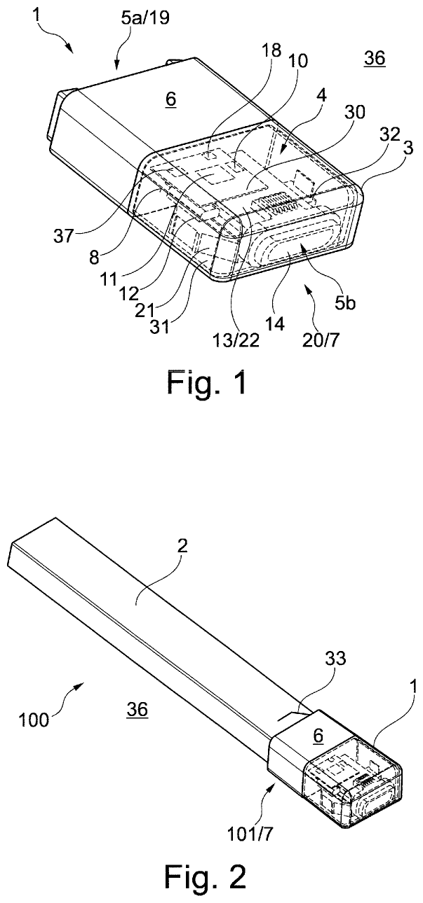

FIG. 1 a perspective view of a mouthpiece;

FIG. 2 a perspective view of an inhalation system;

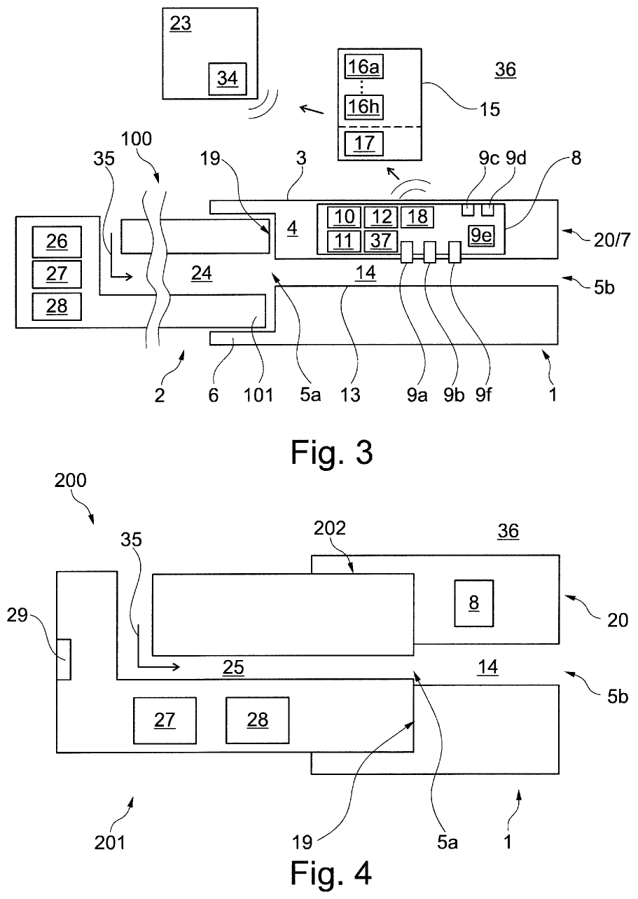

FIG. 3 a schematic sectional view of an inhalation system;

FIG. 4 a schematic sectional view of a cartridge system; and

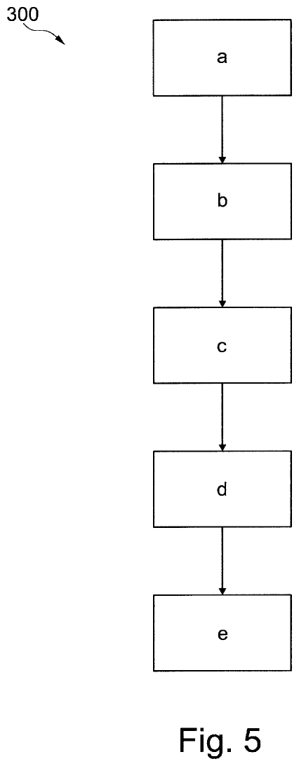

FIG. 5 a schematic representation of a method for determining a system status of an inhaler.

FIG. 1 shows a perspective view of an interchangeable mouthpiece 1 comprising a housing 3 that defines an interior 4 from an environment 36. Furthermore, the housing 4 comprises an air inlet opening 5a and an air outlet opening 5b which are not visible due to the perspective view. The air inlet opening 5a is fluidically connected to the air outlet opening 5b via a flow channel 14. The flow channel 14 extends inside the housing 3 and is separated from the rest of the interior 4 by a partition 13. The air inlet opening 5a and the air outlet opening 5b are arranged on opposite end faces 19 and 20 of the mouthpiece 1, wherein the flow channel 14 extends in a straight line between these end faces 5a and 5b. The air inlet opening 5a is thus connected in a direct path to the air outlet opening 5b.

Furthermore, the mouthpiece 1 comprises an adapter 6 configured to receive an inhaler 2 (see FIG. 2) with its mouth-side end 7 comprising an inhaler mouthpiece 101. The mouthpiece 1 thus replaces, in the connected state, the function of the inhaler mouthpiece 101 held by it via the adapter 6.

Furthermore, a data acquisition device 8 is provided as a component of the mouthpiece 1, which comprises a processor 11, an energy storage device 12, a data storage device 10, a data interface 18 and at least one sensor 9 (see FIG. 3). The processor 11, the data memory 10, and the data interface are arranged on the upper side of a circuit board 30. The energy storage device 12 is arranged on a bottom side of the circuit board 30.

Extending from an inner wall 31 of the housing 3 is a carrier 21 adapted to support the data acquisition device 8 within the housing 3. The carrier 31 supports the data acquisition device 8 via its energy storage device 12. Furthermore, the carrier 21 forms a support for the partition 13 forming the flow channel 14. In addition, the data acquisition device 8 is also supported by a planar outer surface 22 of the partition 13. As can also be seen from FIG. 1, another carrier 32 extends from the inner wall 31 and is also adapted to support the partition 13 forming the flow channel 14. Thus the flow channel 14 is mounted between two carriers 21 and 32. By mounting the flow channel 14 and the data acquisition device 8 in this way in the interior 4 of the housing 3, reliable operation can also be ensured in mobile use.

FIG. 2 shows an inhaler system 100 comprising the inhaler 2, which is connected to the mouthpiece 1 shown in FIG. 1. In FIG. 2, the mouthpiece 1 is in the so-called connected state.

To ensure that the mouthpiece 1 is also secured in the connected state during mobile use, the mouthpiece 1 comprises an additional securing element 33, which prevents the inhaler 2 from slipping out of the adapter 8 by means of a positive connection. The securing element 33 engages in a groove on an outer side of the inhaler 2 for this purpose.

FIG. 3 shows a schematic representation of the inhaler system 100 comprising the inhaler 2 shown in abbreviated form and the mouthpiece 1. FIG. 3 also shows an external evaluation device 23 to which the mouthpiece 1 can transmit the at least one data set 15.

The mouthpiece 1 shown in FIG. 3 corresponds to the mouthpiece 1 of FIG. 1, wherein in the following explanation of the mouthpiece 1 shown in FIG. 3, in order to avoid repetition, only those features will be discussed which are not already apparent from FIG. 1. For example, the energy storage device 12 and the processor 11 are not shown in FIG. 3 for clarity.

The inhaler 2 comprises an inhaler flow channel 24 in which air flows along a flow direction 35 during an inhalation puff at an inhaler mouthpiece 101. The inhaler mouthpiece 101 is adapted to be taken between the lips of a user when the mouthpiece 1 is not attached, so that a negative pressure is created in the inhaler-internal flow channel 24 by sucking on it.

Further, the inhaler 2 comprises an energy storage device 26, a reservoir 28 in the form of a liquid tank containing a liquid to be vaporized, and an adding device 27 in the form of a vaporizer. The adding device 27 is energized by the energy storage device 26 so that the liquid can be vaporized from the reservoir 28. The vaporized liquid is then added to the airflow in the inhaler's own flow channel 24.

The adapter 6 of the mouthpiece 1 engages around the inhaler mouthpiece 101 so that the inhaler 2 with its inhaler mouthpiece 101 projects into the mouthpiece 1 until it comes to rest against an end face 19. Thus, the inhaler mouthpiece 101 is held by the adapter 6 in a predefined position in which it is ensured that the flow channel 24 of the inhaler 2 is coupled with the flow channel 14 of the mouthpiece 1.

The outer surfaces of the inhaler mouthpiece 101 comprise an interference fit with respect to the inner surfaces of the adapter 6, such that the inhaler mouthpiece 101 is retained in the adapter 6 by a force fit connection. The oversize of the inhaler mouthpiece 101 can ensure that the transition between the flow channels 24 and 14 is reliably sealed from the environment.

The flow channel 14 of the mouthpiece 1 extends from an air inlet opening 5a at the end face 19 associated with the adapter 6 to an air outlet opening 5b at an end face 20 of the mouthpiece 1 facing away from the adapter 6. It can further be seen that the flow channel 14 connects the air inlet opening 5a to the air outlet opening 5b in a direct path. Such a design of the flow channel 14 can ensure that approximately the same fluidic conditions prevail in the mouthpiece 1 as in the flow channel 24 of the inhaler 2.

A data acquisition device 4 is further provided within the housing 3, comprising a series of sensors 9a to 9f, which are not shown in FIG. 1 for reasons of clarity.

A pressure sensor 9a, a temperature sensor 9b and a sensor for capturing the flow resistance 9f are arranged in such a way that they are fluidically connected to the flow channel 14 or project into it. By means of these sensors 9a, 9b, 9f, fluid mechanical parameters can be captured within the flow channel 14, for example the air pressure, the flow velocity, the flow resistance and/or the temperature.

Furthermore, in fluidic connection to the environment 36, the ambient temperature sensor 9c is provided. Furthermore, the data acquisition device 8 comprises a GPS receiver 9d and an acceleration sensor 9e.

Alternatively or additionally, further sensors may also be provided, for example a hygrometer, which may then be fluidically connected to the flow channel 14 or to the environment 36, for example.

By means of the sensors 9, operating parameters 16 can be captured, which are then stored on the data memory 10. Furthermore, a hardware clock 37 is provided by means of which the data for a time stamp 17, i.e. date and time, can be generated.

The data acquisition system 8 is adapted to generate and store a data set 15 on the data storage device 10 depending on the fluidic condition in the flow chamber 14. Such a fluidic condition can be, for example, a drop in pressure below a certain value. The drop in pressure can be used in particular to detect the start and end of an inhalation process and thus also to determine the inhalation puff duration 16c as an operating parameter.

Further operating parameters 16, which are stored as part of the data set 15, can be: a mouthpiece ID 16a stored on the data memory 10; an inhalation puff ID 16b generated by the processor 11; an inhalation puff duration 16c; a temperature 16d within the flow channel 14 determined by the temperature sensor 9b; a temperature 16e in the environment 36 of the mouthpiece 1 determined by the ambient temperature sensor 9c; orientation data 16f allowing an inference of the orientation of the mouthpiece 1 in space, determined by the acceleration sensor 9e; coordinates 16g allowing an inference of the position of the mouthpiece 1, determined by the GPS receiver 9d; and/or flow resistance data 16h allowing an inference of the flow resistance in the flow channel 14, determined by the sensor 9f for capturing the flow resistance.

Both the automatic storage of the operating parameters 16 in the form of a data set 15 on the data memory 10 and the provision of this data set 15 with a time stamp 17 are effected by a program code stored on the data memory 10 and executable by the processor 11.

The data acquisition device 8 is adapted to store a plurality of data sets 15 on the data memory 10, preferably several hundred or even several thousand data sets 15. This allows the operating parameters 16 to be stored over a longer period of use up to a life cycle of an inhaler 2. As soon as the data acquisition device 8 is connected to the external evaluation device 23 by means of its data interface 18, all stored data sets 15 can be transmitted to the external evaluation device 23, for example in the form of a data packet.

According to the embodiment shown in FIG. 3, the data set 15 is transmitted directly to the external evaluation device via a Bluetooth module of the data interface 18.

The external evaluation device 23 comprises a receiving interface 34 in the form of a Bluetooth module for receiving the at least one data set 15.

Of course, the data interface 18 may alternatively or additionally comprise other interfaces, such as a contact interface in USB format, a Wi-Fi module and/or a mobile radio module. The receiving interface 34 of the external evaluation device is then also designed in a corresponding manner.

FIG. 4 shows the same mouthpiece 1 as FIG. 3, with the difference that it is connected to a cartridge 201 by means of its adapter 6. The cartridge 201 is usually a component of an inhaler 2 and can be exchangeably inserted into it. Together with the mouthpiece 1, the cartridge 201 forms a cartridge system 200.

The cartridge 201 of FIG. 4 comprises a flow channel 25, which is flowed through during an inhalation puff in flow direction 35. Furthermore, the cartridge 200 comprises an adding device 27 and a reservoir 28, which correspond in design to the adding device 27 and the reservoir 28 of FIG. 3. Power can be supplied to the adding device 27 via an interface 29. The cartridge mouthpiece 202 is received via the adapter 6 in the same way as the inhaler 2 as explained in FIG. 3, so that during an inhalation puff by the user on the mouthpiece 1, the aerosol generated by the adding device 27 flows from the flow channel 25 of the cartridge 201 into the flow channel 14 of the mouthpiece 14.

FIG. 5 shows schematically the sequence of a method 300 for determining a system state of an inhaler 2 comprising the following method steps:

In a process step a), the mouthpiece 1 known from FIGS. 1 to 4 is first mounted on an inhaler mouthpiece 101. Usually, this is done by simply sliding it on.

In a process step b), at least one operating parameter 16 is determined for an inhalation puff and stored as a data set 15 on the data memory 10. One data set 15 is saved on the data memory 10 for each inhalation puff.

In a method step c), a time stamp 17 is assigned to the data set 15 on the data memory 10. The data set 15 thus also comprises the time stamp 17 in addition to the at least one operating parameter 16.

In a method step d), a plurality of stored data sets 15 are transmitted to an external evaluation device 23 by means of the data interface 18.

In a further method step e), the data sets 15 are evaluated by means of the external evaluation device 23. Due to the large number of data sets 15 transmitted, the evaluation can provide statistically significant results.

In a further process step, which follows process step e), the external evaluation device 23 can, for example, display the result of the evaluation, preferably on a display, the result can be transmitted to a further device, or an instruction for action, for example for carrying out a maintenance measure, can be generated based on the result.

As a further process step, this can be followed by the execution of the proposed instruction for action so that the system status of the inhaler can be improved.

As just one example of a system state that can be determined, the total useful life of the inhaler 2 can be calculated by adding up the transmitted values for the inhalation puff duration 16c.

1. An interchangeable mouthpiece for an inhaler, comprising:
a housing which forms an interior and comprises an air inlet opening and an air outlet opening
an adapter via which the mouthpiece is connectable to a mouth-side end of the inhaler so that the air inlet opening is fluidically coupled to the inhaler in a connected state, and
a data acquisition device comprising at least one sensor, a data storage device, a processor and an energy storage device, wherein
a flow channel is formed inside the housing by a partition, fluidically connecting the air inlet opening with the air outlet opening, and wherein the data acquisition device is adapted to store a data set comprising at least one operating parameter on the data memory in dependence of a fluidic condition inside the flow channel.

2. The interchangeable mouthpiece according to claim 1, wherein
the adapter is adapted to be connected to an inhaler mouthpiece.

3. The interchangeable mouthpiece according to claim 1, wherein
the data acquisition device is adapted to detect an inhalation puff based on the fluidic conditions in the flow channel.

4. The interchangeable mouthpiece according to claim 3, wherein
the storing of the data set takes place in dependence on the detection of an inhalation puff.

5. The interchangeable mouthpiece according to claim 1, wherein the data acquisition device comprises one or more of the following sensors:
a pressure sensor for detecting an air pressure in the flow channel,
a temperature sensor for capturing a temperature inside the flow channel
an ambient temperature sensor for capturing the temperature in the environment of the mouthpiece,
a GPS receiver for capturing the current position of the mouthpiece,
an acceleration sensor for capturing the orientation of the mouthpiece in space, and/or
a sensor for capturing the flow resistance in the flow channel.

6. The interchangeable mouthpiece according to claim 1, wherein
the data acquisition device is adapted to provide the data set with a time stamp.

7. The interchangeable mouthpiece according to claim 1, wherein
the stored data set comprises at least one of the following operating parameters:
a mouthpiece ID,
an inhalation puff ID,
an inhalation puff duration,
a temperature within the flow channel,
a temperature in the environment of the mouthpiece,
orientation data, which allow a conclusion about the orientation of the mouthpiece in space,
coordinates, which allow a conclusion to be drawn about the position of the mouthpiece, and
flow resistance data, which allow a conclusion to be drawn about the flow resistance in the flow channel.

8. The interchangeable mouthpiece according to claim 1, wherein
the mouthpiece comprises a data interface for transmitting the at least one stored data set.

9. The interchangeable mouthpiece according to claim 8, wherein
the data interface comprises a wireless interface comprising a Bluetooth module, a mobile radio module and/or a WiFi module.

10. Interchangeable-The interchangeable mouthpiece according to claim 1, wherein
the adapter is formed by a part of the housing.

11. The interchangeable mouthpiece according to claim 1, wherein
the air inlet opening is arranged on a first end face of the mouthpiece associated with the adapter, and
the air outlet opening is arranged on a second mouth-side end face of the mouthpiece facing away from the adapter.

12. The interchangeable mouthpiece according to claim 1, wherein
the flow channel connects the air inlet opening in a direct path to the air outlet opening.

13. A cartridge system comprising a cartridge with its own cartridge mouthpiece, a reservoir with a substance to be vaporized, and an adding device for adding the vaporized substance into an air flow, wherein the cartridge system comprises a mouthpiece according to claim 1, and wherein the adapter of the mouthpiece is detachably connected to the cartridge mouthpiece.

14. An inhaler system comprising an inhaler with its own inhaler mouthpiece a reservoir with a substance to be vaporized, an adding device for adding the vaporized substance into an air stream and an energy storage device, wherein the inhaler system comprises a mouthpiece according to claim 1, and wherein the adapter of the mouthpiece is detachably connected to the inhaler mouthpiece.

15. A method for determining a system state of an inhaler comprising the following method steps:
a) mounting a mouthpiece according to claim 1, on an inhaler's own inhaler mouthpiece;
b) determining and storing at least one operating parameter for an inhalation puff as a data set on the data memory;
c) assigning a time stamp to the data set on the data storage;
d) transmitting a plurality of stored data sets by means of a data interface to an external evaluation device; and
e) evaluating the data sets by means of the external evaluation device.

16. An electronic cigarette product, a conventional cigarette product, or a medical inhaler comprising an interchangeable mouthpiece according to claim 1.
